(19) United States
(12) Patent Application Publication
(10) Pub. No.: US 2006/0147490 A1
(43) Pub. Date: Jul. 6, 2006

(54) New polymers and applications

(76) Inventors: Tim Bowden, Uppsala (SE); Fredrik Nederberg, Stockholm (SE); Jon Hillborn, Sigtuna (SE)

Correspondence Address: YOUNG & THOMPSON, 745 SOUTH 23RD STREET, 2ND FLOOR, ARLINGTON, VA, 22202, US

(21) Appl. No.: 10/526,965

(22) PCT Filed: Sep. 5, 2003
(86) PCT No.: PCT/SE03/01395

§ 371 Date: Sep. 14, 2005

(30) Foreign Application Priority Data
Sep. 5, 2002 (SE) 0202619-3

(51) Int. Cl. C08L 85/02; A61F 2/00

(52) U.S. Cl. 424/426; 525/188

(57) ABSTRACT

The present invention provides a biodegradable, biocompatible polymer that is capable of forming particles (micelles), vesicles, surfaces and membranes, and other structures in which a biologically active agent, e.g. a drug, can be incorporated in such a way that its release to the host can be controlled to a high degree of accuracy, or in where surfaces of the formed polymers can be used to increase the hemeo compatibility of biomaterials. The present invention provides polymer compounds comprising at least one biodegradable polyester having a terminal functional group based on hydrophilic moieties from a phospholipid.

The present invention relates to a novel class of polymers, macro aggregates formed by said polymers, and various uses of said polymers and aggregates for controlled release of substances or to provide temporary coatings to enhance the blood compatibility of biomaterials.

### BACKGROUND OF THE INVENTION

Polymers are a versatile class of materials that offer numerous benefits compared to other material groups. Polymer structures have also been used to facilitate solutions to a variety of biomedical problems. Remarkable properties such as biocompatibility and/or biodegradability, have been the reasons why they have been used in, for instance, sutures and bioactive membranes. Promising future applications involves areas such as platforms for tissue regeneration, stent coatings, replacement materials for eye lenses and various cosmetic solutions.

Another application is the growing need for new sophisticated and “smart” materials for active drug delivery. Controlled drug delivery technology represents one of the most challenging areas of polymer research, and the need for new release systems is high. Such delivery systems offer numerous advantages compared to conventional dosage forms, including improved efficacy, reduced toxicity and improved patient compliance and convenience. Such systems often use synthetic polymers as carriers for the drugs. Although the introduction of the first clinical controlled release systems occurred less then 25 years ago, 1997 sales of advanced drug delivery systems in the United States alone were approximately $14 billion dollars.

The methods of controlled release are generally divided into two classes; temporal control and distribution control. In temporal control, drug delivery systems aim to deliver the drug over an extended duration or at a specific time during treatment. In distribution control, drug delivery systems aim to target the release of the drug to the precise site of activity within the body. The two methods have distinct differences, and in every situation there is a certain need that can be fulfilled depending on the choice of release system.

In order to establish working platforms suitable for either temporal or distribution control release systems, the use of polymers have been widely used. Many polymer classes have been used, including polyesters, polyorthoesters, polyanhydrides, phosphorous containing polymers, and polyamides. Moreover, numerous examples of hydrophobic/hydrophilic block copolymers with surfactant properties have also been made. One example is the polylactic acid (PLA) polyethylene glycol (PEG) copolymer system in which the PEG chain adds hydrophilic properties whereas the PLA chain is hydrophobic, the whole structure being biodegradable. Attempts to target the degradation of the polymer have been made using combinations of phosphoesters and aliphatic polyesters, e.g. U.S. Pat. No. 6,166,173. Furthermore, the use of vesicles has provided an alternative release method and the stability of such systems has been increased, e.g. WO 99/65 466.

A phenomenon often observed with controlled release formulations of medicinal products is that of the “burst effect”, that is, a very large initial release of the active substance. In certain cases, this effect may be desirable. On the other hand, there are cases where it may prove to be dangerous. This is the case, which is particularly detrimental to hormone therapies, which use active principles having very troublesome or even toxic side effects in high concentrations. In such cases, it is imperative to be able to ensure slow and uniform release in small quantities of the active principle.

Attempts to overcome such effects have been made, see e.g. U.S. Pat. No. 6,319,512. The invention claimed in this patent provides an implant for the controlled release of at least one pharmaceutically active agent, said implant comprising a core which contains at least one active agent and a sheath which surrounds said core, and is wherein said sheath is composed of at least one polymeric film applied around said core. According to a preferred embodiment of that invention, the sheath is composed of at least two polymeric films, one surrounding part of the core and the other surrounding the remaining part. This is however, a fairly complex structure, requiring fairly complex manufacturing, and thus has disadvantages in terms of manufacture cost.

Thus, over the last two to three decades there has been a development of controlled release systems for medical use. Numerous patents have been filed and granted on such systems. These systems have been based on various kinds of structures, such as micelles, vesicles, surface bound agents, etc.

Parallel to this development, the need for materials with new biomedical functions has increased, and new areas in which the use of polymers has been introduced are, for instance, bone implant replacements, stent technology, and scaffolds for tissue engineering.

The research groups of Nakabayashi and Ishikara have developed a new type of copolymer in which a hydrophobic polymer has been used in combination with hydrophilic phosphatidyl choline units. By doing so, a new biocompatible amphiphilic structure was created. To mention a few copolymer systems developed by Nakabayashi et al are various polymethacrylates, polysulfones, polyethylenes and polystyrenes, which have been used in combination with phosphatidyl choline units. Some of the most significant improvements compared to the homopolymer have been increased blood compatibility and reduced plasma protein adsorption. These effects have been studied in membranes and surfaces as well as in particles (micelles). It has been shown that the phosphatidyl choline unit may interact with phospholipids to create stable biomembranes as well as an ability of the zwitterionic head group to strongly bind water thereby minimizing the polymer protein interaction leading to increased hemeo compatibility. Since the first published data was released in the early 90's, many other research groups have contributed to further research in the area.

As already indicated, during the last decade polymer research has been driven towards the design of materials with multiple properties. This includes both new polymerization techniques as well as the use of polymers in combination with other highly ordered and controlled structures. The development of dendritic, hyper branched and star-like structures parallel to advances in ring-opening metathesis (ROMP), atom transfer radical (ATRP) and ring-opening (ROP) polymerization techniques have enabled the preparation of well-defined functional polymeric materials with predictable molecular weights and narrow polydispersities. This development has enabled the synthesis of a variety of new architectures developed from a number of different building blocks.

Many of these have been proven successful; however, a biodegradable system with bio-mimicking and non-thrombogenic properties suitable for drug release or enhanced blood compatibility has not yet been developed. This would allow materials to be developed with self-regenerating, anti-fouling surfaces with drug releasing capabilities. Furthermore, introducing “phospholipid-like” analogues with different charged ionic groups would facilitate the combination of specific interactions provided by the charged “phospholipid-like” polymer with the biomimetic polymer that prevent non-specific interaction. Combinations thereof should promote the binding of charged hydrophilic compounds in addition to the incorporation of hydrophobic water-insoluble ones. In addition, this is in similarity to biological environments where for instance the cell membrane-bound phosphatidyl serine has a negative charge. Moreover, the possibility of drugs delivered from loaded particles of such molecules reaching the target cell should increase, thereby facilitating more precise and controlled transport and drug release. This should reduce unwanted side- and release effects. In addition, the degradation leads to products readily metabolized by the human body. Such phospholipid analogues could also enhance the stability of liposomes used in drug delivery or be used in combination with naturally occurring phospholipids to create biomembranes. Applications also include cosmetic formulations.

### SUMMARY OF THE INVENTION

Thus, in view of the need for new materials suitable for controlled release of e.g. medicaments having biodegradable and biocompatible properties, the object of the invention is to make available a biodegradable, biocompatible polymer, with increased blood-compatibility, which is capable of forming particles (micelles), vesicles, surfaces and membranes, and other structures in which a biologically active agent, e.g. a drug, can be incorporated in such a way that its release to the host can be controlled to a high degree of accuracy.

This object is achieved in a first aspect of the invention by a novel polymer compound as defined in claim 1.

In a further aspect there is provided a macromolecule in the form of a self-assembled micelle, dendrimer or membrane structure-based on the polymer defined in claim 1. This macromolecule is defined in claim 2 and claim 3.

There is also provided, in a third aspect of the invention, a vehicle for the controlled release of biologically active agents, e.g. drugs, said vehicle being defined in claim 13. Preferred forms of said vehicle in the form of micelles, vesicles, membranes, and surfaces are defined in the claims depending from claim 13.

Finally, there is also provided a method of making the polymer and polymer aggregates, the method being defined in claims 18-28. The method makes it possible to produce polymers being either anionic, cationic or zwitterionic or neutral, or any combination thereof.

The present invention polymers have several advantages for use in systems for controlled drug release or to provide surfaces with enhanced blood compatibility. One advantage is that, in the present invention the polymers are compatible with blood, a property imparted by the biomimetic phosphatidyl choline (PC). The polymer is also biodegradable. Moreover, the combination of hydrophilic and hydrophobic segments of the material gives the present invention polymers the appropriate physical properties needed to form particles or membranes. In addition, the high level of synthetic control also leads to control of functionality, thereby increasing the flexibility of this new polymer material, i.e. this material makes it possible to incorporate various types of drugs. With the present invention and the accompanying technique for ring-opening polymerization of cyclic esters, it is possible to tailor the length of the polymers or in a later step the size of particles, depending on the application. Particle or membrane formation can be achieved either by self-assembly of linear polymers, or alternatively, by a dendritic approach in order to form a “one molecule-one particle” type of system.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below and the accompanying drawings which are given by way of illustration only, and thus not limitative of the present invention, and wherein

FIG. 1 illustrates the synthetic route for terminated poly ε-caprolactone-phosphatidyl choline (PCL-PC) according to the present invention.

FIG. 2 illustrates micelle formation of an amphiphilic molecule according to the present invention.

FIG. 3 shows an example of a dendrimer structure, a branched polyfunctional one particle molecule, according to the present invention

FIG. 4 exemplifies other cyclic esters that, in addition to ε-caprolactone, that could be used to synthesise the polymer according to the present invention.

FIG. 5 shows a schematic model of possible molecular arrangement in a PCL-PC blend in the form of cast films (left) and after heat treatment in water (right).

FIG. 6 shows a diagram depicting formation of TAT-complex when using a PCL-PC-material in contact with whole blood.

### DETAILED DESCRIPTION OF PREFERRED EMBODIMENTS

The approach has been to combine the use of phosholipid moieties in combination with biodegradable polyesters in order to prepare a fully biocompatible and biodegradable polymer system. One major goal has been to design macromolecules so they form a certain type of structure depending on the application. Two examples are membranes and micelles.

The present invention provides polymer compounds comprising at least one biodegradable polyester having a terminal functional group based on the hydrophilic moiety in phospholipid.

The polymer compounds according to the present invention can be aggregated and have the shape of micelles, vesicles and membranes. The polymer compounds can also be designed such that they emanate from a central core so as to form a dehdrimer. The dendrimer-type of polymer compound forms an essentially spherical particle with said functional groups forming the surface layer of said spherical particle or is concentrated at the surface, thus mimicking the surface of vesicles.

A solution of the micelles or spherical particles formed by the polymer compound according to the present invention can be used as a drug formulation, where the micelles or particles enclose a medicament.

The polymer compound according to the present invention can further be used for coating an object, e.g. a vehicle, and the thus formed coating may be loaded with an (biologically) active agent, e.g. a drug. The coating constitutes a layer having a thickness of 0.1-100 μm, said functional groups forming an outer layer of said coating.

The coated object may be used in biological or medical applications, such as a medical device, medical device for implantation, stent, artificial orthopedic device, spinal implant, joint implant, attachment element, bone nail, bone screw, or a bone reinforcement plate.

The biodegradeable polyester used in the polymer compound according to the present invention is polymerized from a cyclic monomer selected from the group of cyclic esters and carbonates including ε-caprolactone, lactide, glycolide, β-butyrolactone, propiolactone, trimethylenecarbonate and combinations thereof.

The terminal functional group of the polymer compound according to the present invention is positively or negatively charged, or is zwitterionic or electrically neutral. The terminal functional group is selected from but not restricted to phosphatidyl choline, phosphatidyl ethanolamine, phosphatidyl serine, ammonium salt, carboxylic acid or carboxylate, phosphoric acid, phosphate, phosphonate, sulphonate, sulphonic acid, peptide, nucleotide, carbohydrate.

The molecular weight of the polymer compound according to the present invention can be in the range 1000-200 000 g/mol, preferably 20 000 g/mol. The present invention also provides a method of preparing a biodegradable and biocompatible polyester having a terminal functional group based on a phospholipid, which is comprised by the following steps: reacting a cyclic ester monomer and an alcohol in the presence of a catalyst/an initiator to provide a ring opened polymer having an —OH terminal end; reacting the —OH terminal end of the obtained polymer with a phosphorous containing compound to provide a polymer having a phosphate terminated polymer; and reacting said phosphate terminated end of said polymer to obtain a polymer having functionalized end.

The phosphorous containing compound in said method is preferably selected from the group consisting of ethylene chloro phosphates. In said method, the step of providing a functionalized polymer also comprises reacting the terminal end with trimethylamine. The resulting polyester is preferably poly ε-caprolactone-phosphatidyl choline.

The present invention further provides a method of preparing biodegradable and biocompatible polyester amphiphiles having a charged terminal functional group in combination with phosphatidyl choline, the method comprising the following steps: reacting a cyclic ester monomer and an alcohol in the presence of a catalyst/initiator to provide a ring-opened polymer having an —OH terminal end; and reacting said —OH terminal end of the obtained polymer with a co-halo acid halide to obtain an alkyl halide; and reacting said polymer/polymers to obtain a polymer having a functionalized end.

In said method, the step of providing a functionalized polymer comprises reacting the terminal end with trimethylamine. The resulting polyester is preferably poly ε-caprolactone-ammonium salt.

The present invention further provides a method of preparing a biodegradable and biocompatible polyester amphiphiles having a charged terminal functional end in combination with phosphatidyl choline, comprising the steps of reacting a cyclic ester monomer and an alcohol in the presence of a catalyst/an initiator to provide a polymer having an —OH terminal end; reacting the —OH terminal end of the obtained ring-opened polymer with a succinic anhydride to produce a functionalized (carboxylic acid)- or carboxylate-terminated polymer.

In said method, the step of providing a functionalized polymer comprises reacting the terminal end with derivatives of carboxylic acid or its anhydrides. The resulting polyester is preferably poly ε-caprolactone-carboxylic acid or poly ε-caprolactone-carboxylate.

Now the general experimental methods will be described.

Tin(II)triftuoromethane sulfonate (Sn(OTf)₂) was purchased from Aldrich and was azeotropically distilled with toluene prior to use. ε-caprolactone (ε-CL) and triethylamine were purchased from Aldrich and were distilled over calcium hydride prior to use. Chloroform and dichloromethane (VWR) were washed over a basic aluminum oxide (Al₂O₃) column and distilled over CaH₂ prior to use. Succinic anhydride (Aldrich) was recrystallized from dry chloroform and stored in a glove box prior to use. 4-chlorobutyrylchloride (Aldrich) was used as received. Acetonitrile was purchased from Lancaster and was distilled from magnesium sulfate prior to use. Ethylene chloro phosphate was purchased from Lancaster and was distilled and stored in a freezer prior to use. Benzyl alcohol was purchased from Aldrich and was distilled over calciumhydride prior to use. ¹H-NMR and ³¹P-NMR were performed on a JEOL 400 MHz. SEC was performed on a Waters instrument.

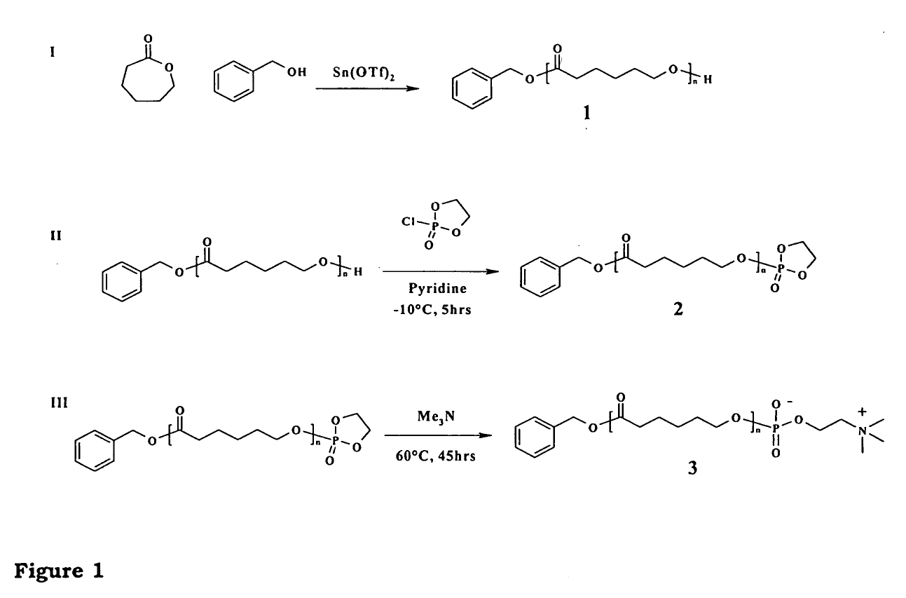

The following section will be based on FIG. 1, which illustrates the synthetic route for terminated poly ε-caprolactone-phosphatidyl choline.

Synthesis of Poly ε-Caprolactone. PCL (Step I in FIG. 1.)

A 50 ml two necked Schlenk flask was added a stir bar and the flask was sealed with a septum. The equipped flask was carefully flame-dried under vacuum and purged with nitrogen. For the polymerization the ε-caprolactone monomer (10.0 g, 87.6 mmol) and the Sn(OTf)₂ catalyst (0.063 g, 0.11 mmol), using 5 mol % to initiator, were added in a glove box. Following removal of the flask the initiator benzyl alcohol (0.23 g, 2.2 mmol for a degree of polymerisation of 40) was syringed into the flask under protecting gas. The mixture was stirred vigorously and rapidly heated to 110° C. Following completion of the reaction (t=60 minutes), the polycaprolactone (PCL) mixture was dissolved in THF and precipitated in 500 ml of cold methanol. The precipitate was filtered and washed repeatedly with methanol and then dried in vacuum at 40° C. until a constant weight was reached.

Synthesis of PCL Coupled to Ethylene Chloro Phosphate (Step II in FIG. 1)

For the phosphorylation 4.0 g (0.86 mmol) of a PCL with a degree of polymerisation (DP) of 40 was weighed in a pre-dried nitrogen flask and dissolved in 20 ml dry dichloromethane (CH₂Cl₂). 1.5 equivalents of dry pyridine (0.11 ml, 1.29 mmol) were thereafter added under nitrogen. The flask was attached to a pre dried dropping funnel and attached to a nitrogen inlet and thereafter cooled to −5° C. 5 mL of dry CH₂Cl₂ and 2 equivalents of ethylene chloro phosphate (0.14 g, 1.028 mmol) was added the dropping funnel. The solution was slowly added drop wise and the solution was stirred for approximately 2 hours and then slowly allowed to reach ambient temperature and further stirring for 4 hours. After the reaction was completed the solution was charged with an additional 50 ml of CH₂Cl₂ and extracted twice with distilled water (50 ml) and twice with a 1 M NaHCO₃ (50 ml) solution to remove the, from the reaction formed, pyridinium salt and excess ethylene chloro phosphate reagent. Thereafter the organic phase was separated and dried from water using sodium sulphate, stirring for 30 minutes. 50 ml of toluene was added and the organic phases as well as trace amounts of pyridine was removed by rotational evaporation at ambient temperature.

Synthesis for the Ring-Opening of Ethylene Phosphate to Yield Phosphatidyl Choline Terminated PCL (Step III in FIG. 1)

For the formation of PC terminated PCL 1.0 g (0.21 mmol) of 2 was weighed in a 50 mL pre-dried round-bottom flask and thereafter dissolved in 10 ml of dry acetonitrile. The solution was transferred to a pressure tube with two stopcocks, purged with nitrogen and sealed, thereafter cooled to −10° C. Two equivalents (0.42 mmol, 39 μl) of trimethylaminew to the PCL polymer was carefully condensed into the pressure tube and thereafter slowly heated to 60° C. The pressure tube was left under stirring for 45 hours and then left to cool to ambient temperature and the reaction product was precipitated in cold methanol. The precipitate was collected and dried until constant weight.

Synthesis of PCL Coupled to Succinic Anhydride

For the synthesis 2.0 g (0.44 mmol) of 1 and 88 mg (0.88 mmol) of succinic anhydride was added a 50 ml pre dried two-necked round bottom flask equipped with a stir bar and purged with nitrogen. The compounds were dissolved in 15 ml of dry chloroform and a dropping funnel was attached and the solution cooled to 0° C. 86 mg (0.88 mmol) of tri ethylamine was added 5 ml of dry chloroform and charged in the funnel and slowly added drop wise to the cooled solution under a 30-minute period. The solution was slowly allowed to reach ambient temperature and left under stirring for an additional 3 hours. Following complete conversion the polymer was precipitated in cold methanol filtrated and dried until constant weight.

Synthesis of PCL with a Terminal Quaternary Ammonium

For the synthesis 2.0 g (0.44 mmol) of 1 and 87 mg (1.10 mmol) of pyridine was added a 50 ml pre dried two-necked round bottom flask equipped with a stir bar and purged with nitrogen. The compounds were dissolved in 15 ml of dry chloroform and a dropping funnel was attached and the solution cooled to −10° C. 116 mg (1.10 mmol) of 4-chlorobutyryl chloride was added 5 ml of dry chloroform and charged in the funnel and slowly added drop wise to the cooled solution under a 30-minute period. The solution was slowly allowed to reach ambient temperature and left under stirring for an additional 3 hours. Following complete conversion the polymer was precipitated in cold methanol filtrated and dried until constant weight. The precipitate was thereafter dissolved in 10 ml of dry acetonitrile. The solution was transferred to a pressure tube with two stopcocks, purged with nitrogen and sealed, thereafter cooled to −10° C. Two equivalents (0.42 mmol, 39 μl) of trimethylamine_{(g)} to the PCL polymer was carefully condensed into the pressure tube and thereafter slowly heated to 60° C. The pressure tube was left under stirring for 45 hours and then left to cool to ambient temperature. The formed compound was precipitated in cold methanol and the precipitate was collected and dried until constant weight.

Results

The emphasis was to synthesise a fully biodegradable polymer in combination with phosphatidyl choline as a possible future carrier for controlled drug release or a temporarily coating for enhanced blood compatability or other biomedical applications. The ambition has been to introduce the use of phospholipid analogues into new areas of polymer research, keeping in mind what already has been done in this area. With the recent development of polymerisation techniques for the synthesis of biodegradable polyesters it is not until now that this has been possible. Controlled ring-opening polymerisation of for instance lactides and ε-CL now makes it possible to design polyesters with controlled molecular weight and narrow polydispersities. It should also be pointed out that by the approval from the food and drug administration (FDA), both PCL and PLA are classified as biocompatible polymers, which degrades into molecules acceptable in the human metabolism.

Synthesis:

In our initial results a series of various linear PCL with various molecular weights was made, mainly to demonstrate the high level of control in this synthetic route, but also to create the first amphiphiles with particle or membrane forming properties. The following table recall some initial polymerisation data.

TABLE 1
Ring-opening polymerisation data of PCL.

| | | | Temp. | Time | Yield | I/M | | |
|---|---|---|---|---|---|---|---|---|
| Sample | Catalyst | Initiator | [° C.] | [hrs] | [%] | Ratio | DP | PDI |
| 1 | Sn(OTf)₂ | EtOH | 35 | 4 | 70 | 5 | 5 | 1.14 |
| 2 | Sn(OTf)₂ | EtOH | 35 | 15 | 90 | 15 | 17 | 1.18 |
| 3 | Sn(OTf)₂ | EtOH | 35 | 20 | 95 | 30 | 27 | 1.07 |
| 4 | Sn(OTf)₂ | EtOH | 35 | 39 | 97 | 60 | 66 | 1.19 |

From Table 1 it is clear that the molecular weight of PCL can be controlled by the ratio of initiator to monomer, as previously explained. Using ¹H-NMR analysis the PCL was fully characterized and both the α- and the ω-end groups were identified. The following chemical shifts were observed for the PCL molecule initiated from benzyl alcohol: ¹H-NMR (CDCl₃) δ=1.35 (m, 2H, —CH₂—, poly), 1.65 (m, 2H, —CH₂—, poly), 1.65 (m, 2H, —CH₂—, poly), 2.30 (t, 2H, —CH₂—, poly), 3.63 (q, 2H, —CH₂—, ω-end), 4.04 (t, 2H, —CH₂—, poly), 5.10 (s, 2H, —CH₂—, α-end), 7.34 (m, 5H, —ArH, α-end)

¹H-NMR analysis could be used to monitor the transformation of hydroxyl to ethylene phosphate as the proton group adjacent the hydroxyl group at 3.62 ppm was diminished, while at the same time a build up of resonance's from the ethylene protons in the phosphate was observed at 4.32 ppm. Furthermore, ³¹P-NMR analysis provided a second spectroscopy analysis to track the formation of the ethylene phosphate terminated PCL as the ³¹P-NMR signal of the starting material was shifted from 23.1 ppm to 18.0 ppm in the case of ethylene phosphate. ¹H-NMR (CDCl₃) δ=1.35 (m, 2H, —CH₂—, poly), 1.63 (m, 2H, —CH₂—, poly), 1.63 (m, 2H, —CH₂—, poly), 2.30 (t, 2H, —CH₂—, poly), 4.04 (t, 2H, —CH₂—, poly), 4.32-4.48 (m, 4H, —CH₂—CH2—, ω-end), 5.10 (s, 2H, —CH₂—, α-end), 7.34 (m, 5H, —ArH, α-end) ³¹P-NMR (CDCl₃) δ=18.3

In the last ring-opening step, the final PCL phospatidyl choline molecule was also characterised with ¹H-NMR. A distinct singlet from the methylene signals in the choline unit was observed at 3.42 ppm. The ethylene protons in the phosphatidyl unit are now separated at 3.75 ppm and at 4.20 ppm. ³¹P-NMR analysis revealed the phosphorous signal from the PC group at −1.1 ppm compared to the intermediate phosphorous signal at 18.0 ppm. From the ¹H- and ³¹P-NMR results, it is clear that the synthetic route is functioning. Importantly the synthesis could be performed with complete conversion between each step and with high yields, typically around 90% of PCL-PC.

Having established a synthetic route for the “phospholipid-like” PCL-PC polymer the scope of the synthesis was enhanced to also include charged “phospholipid-like” polymers having a net anionic or cationic charge. To provide a phospholipid analogue with a net anionic charge a PCL with a terminal hydroxyl group was reacted with succinic an hydride in the presence of triethyl amine resulting in the wanted terminal succinic acid monoester. ¹H-NMR analysis was used to monitor the conversion and a build up of the succinate protons, i.e. two triplets, were formed at 2.65 ppm whereas the ethylene protons adjacent the hydroxyl was shifted to 4.12 ppm.

¹H-NMR (CDCl₃) δ=1.35 (m, 2H, —CH₂—, poly), 1.65 (m, 2H, —CH₂—, poly), 1.65 (m, 2H, —CH₂—, poly), 2.30 (t, 2H, —CH₂—, poly), 2.62 (t, 2H, —CH₂—, ω-end), 2.62 (t, 2H, —CH₂—, ω-end), 4.04 (t, 2H, —CH₂—, poly), 5.10 (s, 2H, —CH₂—, α-end), 7.34 (m, 5H, —ArH, α-end)

For the formation of a cationic phospholipid analogue the synthesis was somewhat more complex and consists of two separate steps. In the first step 4-chlorobutyryl chloride was reacted with the terminal hydroxyl group of the polymer. Following purification the intermediate was redissolved in acetonitrile and reacted with Me₃N at 60° C. to allow formation of the cationic quaternary ammonium salt with the chloride ion as gegen ion. ¹H-NMR was used to characterize the obtained product and the methyl resonance of the quaternary ammonium salt was observed at 3.43 ppm. Furthermore, the proton group adjacent the quatemary ammonium was observed at 3.72 ppm.

¹H-NMR (CDCl₃) δ=1.35 (m, 2H, —CH₂—, poly), 1.65 (m, 2H, —CH₂—, poly), 1.65 (m, 2H, —CH₂—, poly), 2.10 (m, 2H, —CH₂—, ω-end), 2.30 (t, 2H, —CH₂—, poly), 2.50 (t, 2H, —CH₂—, ω-end), 3.43 (s, 9H, —CH₃ , ω-end), 3.72 (t, 2H, —CH₂—, ω-end), 4.04 (t, 2H, —CH₂—, poly), 5.10 (s, 2H, —CH₂—, α-end), 7.34 (m, 5H, —ArH, α-end)

Particle Formation:

Following the synthesis two particle formation experiments were performed, mainly to get an indication on how these structures behaved. Particle formation using two different routes was conducted.

Using the first route, a phosphatidyl choline terminated PCL (DP=16) was dissolved in chloroform (CHCl₃). Thereafter the dissolved compound was added drop wise to water. Well-defined drops were formed (two-phase system). Following addition, a stir bar was added and stirring was applied for approximately 30 minutes, creating a fine-dispersed particle solution. In the early stage, after the stirring had been stopped, flocculation was observed. However, after 30 minutes of stirring only stable particles were obtained. By “stable” it is meant that no visual flocculation occurred, indicating stable particles. Environmental-Scanning Electron Microscopy (E-SEM) analysis indicated particles with a diameter of 1-10 μm. Evaporation of the chloroform solidified the particles.

Using the second route, a solvent combination was chosen that could allow a single phase of a combination of the solvents but with the PCL-phosphatidyl choline being totally soluble in one. An acetone-water combination was chosen (5 mL/95 mL) and a small amount (10 mg) of the PCL (DP=16)-phosphatidyl choline. At first the compound was dissolved in acetone, and was thereafter added drop wise into water. After the addition, the solution was perfectly transparent, indicating particle sizes in the nanometer (nm) range.

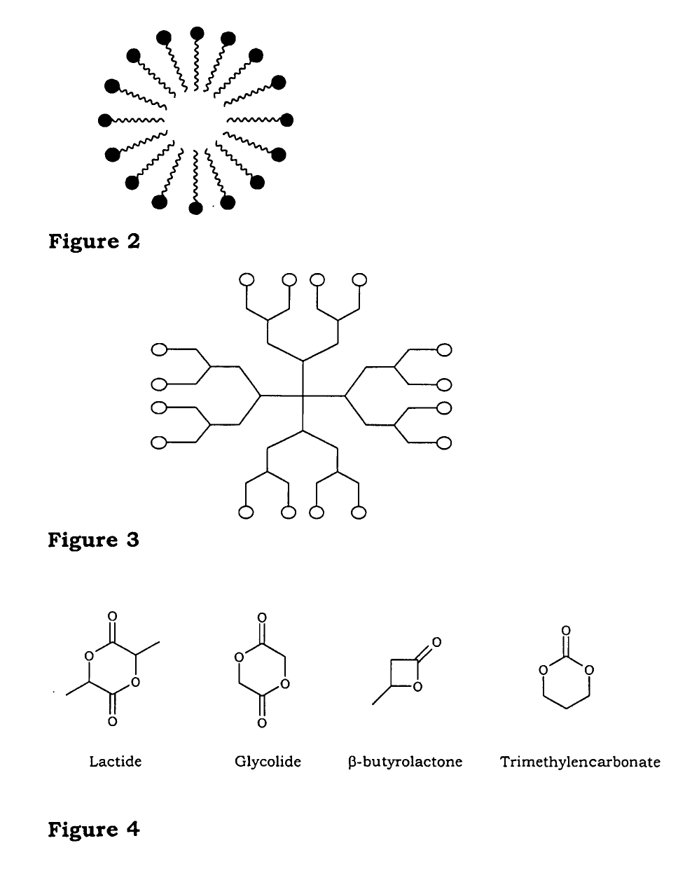

These two experiments gave an indication on that particle formation is possible and that the system is surface-active. The desired effect is seen in FIG. 2, which shows the micelle formation of the amphiphilic molecule.

In FIG. 2 the rings represent hydrophilic phosphatidyl choline units whereas the zigzag lines represent hydrophobic PCL chains. The figure schematically shows the self-assembly of these molecules in an aqueous medium (please observe that the rings could mean an end group which is not phosphatidyl choline, i.e. anionic or cationic in combination with PC).

Film Properties:

The mechanism explaining the low protein adsorption and cell attachment for non-degradable phosphatidylcholine functional polymers involves both surface-enrichment of the phosphatidylcholine unit and the attraction of phospholipids to this surface to form a biomembrane-like structure. It is therefore conceivable that the biodegradable amphiphile PCL-PC could function the same way in addition to being biodegradable.

Films of the above PCL-PC oligomers were not stable in water. PCL (M_{w}˜80 000 g/mol) blend with PCL-PC were good film-formers and could be cast into homogenous films. The surface composition of as cast films of PCL/PCL-PC (DP=45) was quite similar to pure PCL as shown by XPS with a C/O ratio of 73/27 with no signs of phosphor or nitrogen. Contact angle of cast PCL/PC films was 65 degrees, which is only slightly lower than the 69 degrees measured on pure PCL. This is not surprising, considering the low content of phosphatidylcholine end groups and the hydrophobic nature of PCL. As the system strives to minimize its interfacial energy, the phosphatidylcholine chain ends will be buried in the bulk exposing pure PCL to the polymer-air interface.

Under water, however, interfacial free energy is minimized when the hydrophilic phosphatidylcholine would be enriched at the polymer-water interface. Therefore, the PCL/PCL-PC blend film was quickly immersed into heated water at 90° C. (which is above the melting temperature for PCL) to provide molecular mobility for migration. The film first became transparent due to melting of the crystalline PCL. Prior to cooling, the film again became opaque due to water uptake by micellar domains of phosphatidylcholine in the bulk at 90° C. During cooling further opaqueness occurred as the polymer recrystallised.

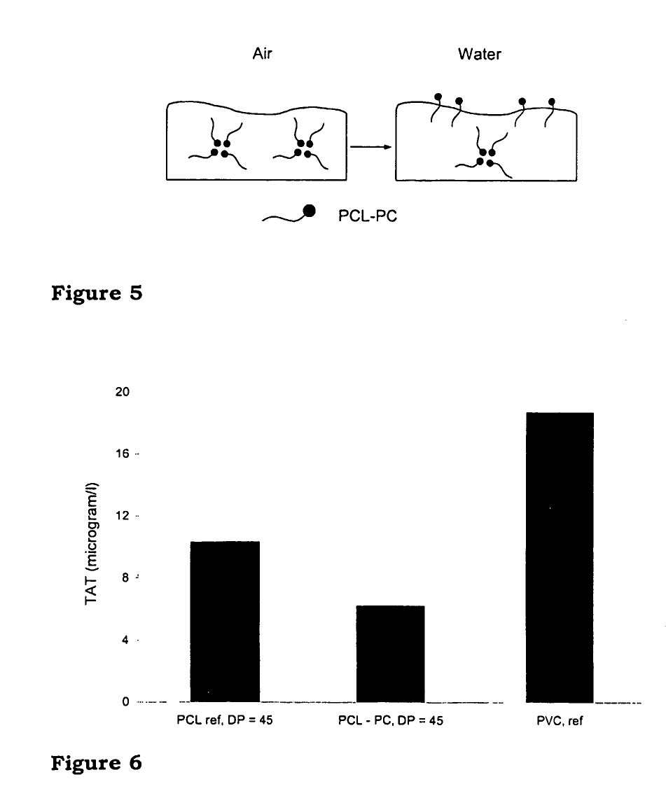

Migration of PCL-PC oligomers to the surface was indeed confirmed by contact angle measurements. The contact angle (advancing) was decreased to 40 degrees, which is indicative of enrichment of polar groups at the surface towards water. ESCA spectrum shown in FIG. 5 reveals the appearance of both nitrogen, N1s 2.4%, and of phosphorous, P2p 1.5% arising from the polar, surface-oriented, phosphatidylcholine. The theoretical concentration in pure PCL-PC (DP=45) is only 0.3%.

It is likely that some surface rearrangement still occurs in the amorphous top layer when the samples are dried prior to ESCA and contact angle measurements. Surface dynamics should therefore be investigated further using dynamic contact angle. The overall mechanism is summarized in FIG. 5 where PCL is oriented towards the air surface with PC forming micellar domains in the bulk of cast films. Upon heating in water, however, the surface rearranges to drive PC towards the polymer-water interface.

Whole Blood Measurements:

Since the fate of biomaterials is strongly dependent on the activation of the blood plasma cascade system, like the coagulation system, the formation of thrombin-anti-thrombin (TAT) complexes were studied in the slide chamber model. The slide chamber methodology facilitates in vitro analysis of biomaterial surfaces in contact with whole blood. A PCL-PC system with a DP of 45 was used as well as two reference surfaces consisting of PCL and polyvinylchloride (PVC). A diagram depicting TAT formation is shown in FIG. 6. This result indicates that the PCL-PC system holds non-thrombogenic properties and that the formation of TAT is largely reduced compared to both PCL and PVC, a well-known biomaterial. This effect can be explained by the enrichment of PC groups on the polar surface, which decreases adherence of proteins. Moreover, the thrombocyte count was larger for whole blood in contact with the PCL-PC surface than the PVC reference

Molecular Variation:

To extend the use of this synthetic route, one can also include non-linear type of molecules. In a totally branched system, e.g. initiated from a polyol or macro initiator, one could obtain a “one-molecule-one-particle” system, in which the self-assembly from many molecules has been changed into a “one-molecule-one-particle” forming system with a controlled size. Dendritic type of structures could for instance be synthesized from the coupling of benzylidine protected bis(hydroxymethyl) propionic acid (bis-MPA) with benzyl protected bis-MPA followed by selective deprotection to yield a first generation dendrimer. An alternative approach would be the tailoring of a dendritic structure from benzylidene-protected glycerol and 2-bromopropionic acid. The addition of branching points in combination with ring-opening polymerization gives an almost unlimited source of architectural possibilities, and the common link is that the functionality on the surface will be much higher compared to linear structures. The hydrophobic unit would still be biodegradable polyester, and the phopshatidyl choline unit imparting hydrophilic properties. One change is the architecture of the molecule, i.e. the branching points, which yield a molecule with a much higher functionality on the surface. The end-functionality, however, does not always have to be phosphatidyl choline; other functionalities or combinations of functionalities can also be chosen, to add specific interactions, as well as the addition of for example receptor ligands. With this synthetic approach, structure, size and functionality can be controlled. One visual example of such a structure is a branched polyfunctional one particle molecule, as seen in FIG. 3 (please observe that the rings could mean an end group which is not phosphatidyl choline).

The monomer used in the previously described synthetic routes has in all cases been ε-CL. Recently, the controlled ring-opening polymerization of other cyclic esters has been investigated, and it is now possible to tailor the molecular weight of other polyesters as well. A summary of other cyclic esters and carbonates, which either separately or in combination, could be used in the described synthesis, is shown in FIG. 4. In all cases the, obtained polyester is biodegradable.

Thus, according to the present invention, a fully biodegradable polyester-phosphatidyl choline compound was synthesized using highly developed polymerization techniques. This molecule had amphiphilic behavior due to hydrophobic properties from the PCL chain and hydrophilic properties from the phosphatidyl choline unit. PCL is one example of a biodegradable polyester, but according to the present invention other monomers, such as lactides, could also be used to produce similar structures. In the present invention synthetic route, only a linear type of molecules was created, but it is also possible to provide branched/dendritic type of structures with a much higher functionality on the surface. The polymers according to the present invention can suitably be used in biological and medical applications, for instance as membranes and as drug delivery vectors.

With the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A polymer compound, comprising at least one biodegradable polyester having a terminal functional group based on hydrophilic moieties of phospholipids.

2. A polymer compound as claimed in claim 1, comprising a plurality of biodegradable polymers emanating from a central core so as to form a dendrimer.

3. Aggregate of polymers as claimed in claim 1, having the shape of micelles, vesicles and membranes.

4. A polymer compound as claimed in claim 1, wherein said polyester is polymerized from a cyclic monomer.

5. A polymer compound as claimed in claim 4, wherein said cyclic monomer is selected from the group of cyclic esters and carbonates.

6. A polymer compound as claimed in claim 5, wherein said cyclic esters and carbonates are selected from the group consisting of ε-caprolactone, lactide, glycolide, β-butyrolactone, propiolactone, trimethylenecarbonate and combinations thereof.

7. A polymer compound as claimed in claim 1, wherein the terminal functional group is phosphatidyl choline, phosphatidyl ethanolamine, phosphatidyl serine, ammonium salt, carboxylic acid or carboxylate, phosphonic acid, phosphate, phosphonate, sulphonate, sulphonic acid, peptide, nucleotide, carbohydrate.

8. A polymer compound as claimed in claim 1, wherein the terminal functional group is positively charged.

9. A polymer compound as claimed in claim 1, wherein the terminal functional group is negatively charged.

10. A polymer compound as claimed in claim 1, wherein the terminal functional group is zwitterionic or electrically neutral.

11. A polymer compound as claimed in claim 1, the molecular weight of which is in the range of 1000-200 000 g/mol, preferably 20 000 g/mol.

12. A dendrimer type polymer compound as claimed in claim 2, forming an essentially spherical particle with said functional groups forming the surface layer of said spherical particle.

13. An object provided with a coating made of a polymer compound as claimed in claim 1, wherein said polymer compound forms a layer having a thickness of 0.1-100 μm, said functional groups forming an outer layer of said coating.

14. The object as claimed in claim 13, wherein said coating is loaded with an (biologically) active agent.

15. The object as claimed in claim 13, wherein the object is an object used in biological or medical applications.

16. The object as claimed in claim 15, wherein it is a medical device, medical device for implantation, stent, artificial orthopedic device, spinal implant, joint implant, attachment element, bone nail, bone screw, or a bone reinforcement plate.

17. A drug formulation, comprising a solution of micelles or spherical particles formed by a polymer compound as claimed in claim 1, wherein the micelles or particles enclose a medicament.

18. A method of preparing a biodegradable and biocompatible polyester having a terminal functional group based on a phospholipid, the method comprising the following steps:
reacting a cyclic ester monomer and an alcohol in the presence of a catalyst/an initiator to provide a ring opened polymer having an —OH terminal end;
reacting the —OH terminal end of the obtained polymer with a phosphorous-containing compound to provide a polymer having a phosphate terminated polymer; and
reacting said phosphate terminated end of said polymer to obtain a polymer having functionalized end.

19. The method as claimed in claim 18, wherein said phosphorous containing compound is selected from the group consisting of ethylene chloro phosphate.

20. The method as claimed in claim 18, wherein the step of providing a functionalized polymer comprises reacting the terminal end with Me₃N.

21. The method as claimed in claim 18, wherein the resulting polyester is poly ε-caprolactone-phosphatidyl choline.

22. The method as claimed in claim 21, wherein the resulting yield of the poly E-caprolactone-phosphatidyl choline is at least 90%.

23. A method of preparing biodegradable and biocompatible polyester phospholipid-like analogues having a cationic terminal functional group, the method comprising the following steps:
reacting a cyclic ester monomer and an alcohol in the presence of a catalyst/an initiator to provide a ring-opened polymer having an —OH terminal end;
reacting said —OH terminal end of the obtained polymer with a ω-halo acid halide to obtain an alkyl halide; and
reacting said polymer/polymers to obtain a polymer having a functionalized end.

24. The method as claimed in claim 23, wherein the step of providing a functionalized polymer comprises reacting the terminal end with Me₃N.

25. The method as claimed in claim 23, wherein the resulting polyester is poly ε-caprolactone-ammonium salt.

26. A method of preparing a biodegradable and biocompatible polyester phospholipid-like analogues having an anionic terminal functional group, the method comprising the following steps:
reacting a cyclic ester monomer and an alcohol in the presence of a catalyst/an initiator to provide a polymer having an —OH terminal end; and
reacting the —OH terminal end of the obtained ring-opened polymer with a succinic anhydride to produce a functionalized (carboxylic acid)- or carboxylate-terminated polymer.

27. The method as claimed in claim 26, wherein the step of providing a functionalized polymer comprises reacting the terminal end with derivatives of derivatives of carboxylic acid or its anhydrides.

28. The method as claimed in claim 26, wherein the resulting polyester is poly ε-caprolactone-carboxylic acid or poly ε-caprolactone-carboxylate.
